(19) United States
(12) Patent Application Publication
(10) Pub. No.: US 2026/0273116 A1
(43) Pub. Date: Sep. 17, 2026

(54) Apparatus for sterilizing containers, apparatus for transporting containers, holding device for containers and method

(71) Applicant: KRONES AG, Neutraubling (DE)

(72) Inventors: Florian GELTINGER, Donaustauf (DE); Thomas SPITZER, Wiesent (DE); Norbert KINDL, Tegernheim (DE)

(21) Appl. No.: 19/566,700

(22) Filed: Mar. 13, 2026

(30) Foreign Application Priority Data
Mar. 13, 2025 (DE) 102025109606.6

(51) Int. Cl. A61L 2/16; A61L 2/08; A61L 2/26

(52) U.S. Cl. CPC A61L 2/16; A61L 2/08; A61L 2/26; A61L 2202/122

(57) ABSTRACT

An apparatus for treating plastics material preforms includes a transport device, at least one application device for applying a flowable sterilization medium to the plastics material containers transported by the transport device, wherein the transport device has a movable transport carrier that is rotatable about a predefined axis of rotation and on which a plurality of holding devices for holding the plastics material containers. The holding device has a gripping device which for contacting the plastics material containers at least in a first portion of the plastics material containers, and a retaining device for contacting the plastics material containers in a second portion of the plastics material containers, wherein the first portion is spaced apart from the second portion, wherein the gripping device is fixed relative to the transport device and the retaining device is movable relative to the transport device.

### CROSS REFERENCE TO RELATED APPLICATION

The subject application claims benefit to German Patent Application Serial no. 10 2025 109 606.6, filed March 13, 2025, the contents of which are incorporated herein by reference.

### BACKGROUND OF THE INVENTION

The present invention relates to an apparatus and to a method for treating and in particular sterilizing containers, in particular plastics material containers, in particular plastics material preforms. In addition, the present invention also relates to a transport device for transporting plastics material containers, in particular plastics material preforms, as well as to a holding device for holding plastics material containers, in particular plastics material preforms.

Different apparatuses and methods for sterilizing plastics material preforms are known from the prior art. Typically, when producing plastics material bottles, a plastics material preform is first heated and then transformed into the plastics bottle, such as a beverage container, using a forming device such as a stretch blow molding machine.

It is also known in the prior art that the plastics material preforms are sterilized and a sterilization agent such as (in particular gaseous) hydrogen peroxide is in particular applied thereto in order to sterilize them. However, sterilization by electromagnetic radiation is also conceivable, to which the present invention is also applicable in principle.

For this purpose, sterilization modules are known in the prior art. For example, the applicant’s prior art discloses a sterilization module with three treatment starwheels, each of which has handling clamps for gripping the containers. It should be noted that, within the scope of the present description, the plastics material preforms are in particular also considered to be containers.

Due to increasing output and treatment times or log rates, significantly more of these types of handling clamps are needed. This results in increased costs, and commissioning and maintenance also become more complex.

The object of the present invention is therefore providing a sterilization apparatus and a method that allows higher output and greater treatment times. In addition, a transport device for transporting containers, in particular plastics material preforms, is also to be provided, which will allow for higher throughput. In addition, a holding device for holding containers, in particular plastics material preforms, is also to be provided, which will also allow for higher throughput.

Transport devices are also known in the prior art which, for example, have sawtooth starwheels, wherein each recess of such a sawtooth starwheel carries a plastics material preform. To prevent the plastics material preforms from falling out of the sawtooth starwheel or the recesses, a guide arch is provided that is arranged outside the sawtooth starwheel. However, this partially hinders access to the containers or plastics material preforms, in particular if they are to be sterilized, for example.

These transport devices still have the disadvantage that they become unreliable, in particular at high transport speeds. A further object of the invention is therefore to improve the reliability of such transport devices.

### SUMMARY OF THE INVENTION

An apparatus according to the invention for treating containers and in particular for sterilizing containers, in particular plastics material containers and in particular plastics material preforms, has a transport device for transporting the plastics material containers along a predefined transport path. Furthermore, the apparatus preferably has at least one application device suitable and intended to apply a flowable and in particular gaseous sterilization medium (or optionally electromagnetic radiation) and/or a coating and/or electromagnetic radiation to the plastics material containers transported by the transport device, wherein the transport device has a movable transport carrier that is preferably rotatable about a predefined axis of rotation and on which a plurality of holding devices for holding the plastics material containers are arranged. Instead of the application device, other treatment devices could also be provided which carry out treatments on the plastics material containers.

The invention will be described with reference to a treatment process in the form of a sterilization process. The invention is also particularly advantageously suited for this purpose. However, other treatment processes would also be conceivable, such as rinsing processes, printing processes, coating processes, heating processes, or cooling processes. However, these are in particular treatment processes that treat at least a portion of the plastics material container and/or plastics material preform.

According to the invention, the holding device or each of the holding devices has/have a gripping device which is suitable and intended for contacting the plastics material containers at least in a first portion of the plastics material containers.

Furthermore, the holding device or each of the holding devices has/have retaining devices, which are suitable and intended for contacting the plastics material containers in a second portion of the plastics material containers, wherein the first portion is spaced apart from the second portion, wherein the gripping device is fixed relative to the transport device and the retaining device is movable relative to the transport device.

The gripping device can be, for example, a (first) gripping arm of a gripping clamp. The retaining device can, for example, also act like a second gripping arm of a gripping clamp.

Preferably, the retaining device is configured in such a way that it can retain the plastics material container and/or prevent the plastics material container from releasing from the gripping device.

Preferably, the first portion is spaced apart from the second portion in a circumferential direction of the plastics material containers.

Preferably, the gripping device surrounds the plastics material container to be held and/or the first portion to be held at a first predefined circumferential angle, and the retaining device surrounds the plastics material container to be held, in particular the second portion, at a second predefined circumferential angle.

Preferably, the second circumferential angle is larger than the first circumferential angle.

Preferably the first circumferential angle is greater than 20°, preferably greater than 30°, preferably greater than 40°, preferably greater than 50°, preferably greater than 60°, preferably greater than 70°, and preferably greater than 80° (in particular with respect to the longitudinal direction of the plastics material container to be held).

The first circumferential angle is preferably smaller than 150°, preferably smaller than 140°, preferably smaller than 130°, preferably smaller than 120°, preferably smaller than 110°, preferably smaller than 100° (in particular with regard to the longitudinal direction of the plastics material container to be held).

Preferably the second circumferential angle is greater than 40°, preferably greater than 50°, preferably greater than 60°, preferably greater than 70°, preferably greater than 80°, preferably greater than 90°, preferably greater than 100° and preferably greater than 120° (in particular with respect to the longitudinal direction of the plastics material container to be held).

The second circumferential angle is preferably smaller than 170°, preferably smaller than 160°, preferably smaller than 150°, preferably smaller than 140° and preferably smaller than 130° (in particular with regard to the longitudinal direction of the plastics material container to be held).

Preferably, the first circumferential angle and the second circumscribed angle together are greater than 180°, preferably greater than 190°, preferably greater than 200°, and particularly preferably greater than 210°, and particularly preferably greater than 220°, and particularly preferably greater than 230°. Preferably, the sum of the first circumferential angle and the second inscribed angle is less than 320°, preferably less than 310°, preferably less than 300° and particularly preferably less than 290°.

However, it would also additionally or alternatively be conceivable for the second portion to be spaced apart from the first portion in a longitudinal direction of the plastics material containers.

In a further embodiment, it would also be possible to provide a gripping device having at least two gripping arms (which, however, can also be integral with one another) in addition to the retaining device, wherein at least a first portion of the containers, in particular plastics material preforms, can be received between these gripping arms, in particular in a recess formed between these two gripping arms.

Two variations are described below. In both embodiments, a gripping device having at least one gripping element (or gripping arm) and a retaining device is provided, wherein the gripping element is fixed relative to the transport device (and/or the plastics material container and/or plastics material preform to be gripped) and the retaining device is movable relative to the transport device (and/or the plastics material container and/or plastics material preform to be gripped) and is in particular pivotable with respect to a predefined pivot axis.

In a first embodiment, a gripping element or gripping device is fixed and a retaining device is movable (with respect to the transport device and/or the plastics material container).

The retaining device preferably has an undercut or recess which, in a retaining position, holds the plastics material container or plastics material preform in a second portion. Preferably, a pivot axis of the retaining device extends in parallel with a longitudinal direction of the plastics material preform. This embodiment is explained in FIG. 3 below.

Preferably, the gripping device does not form an undercut.

In a further embodiment, a gripping device forms (in particular by two gripping arms) a pocket for receiving the plastics material containers. Preferably, the retaining device contacts the plastics material container at a portion that is spaced apart from the first portion in the longitudinal direction. Particularly preferably, a pivot axis of the retaining device is perpendicular to the longitudinal direction of the plastics material containers or plastics material preforms. This embodiment is explained in FIG. 2 below.

These two embodiments are explained in more detail below.

The longitudinal direction or longitudinal axis is in particular an axis which extends from the mouth of the container to the bottom of the container (and in particular to a dome at the bottom of a plastics material preform).

This prevents the plastics material containers from falling out of the recess formed between the gripping arms. Therefore, the retaining device is preferably also a component of the holding devices. Preferably, each holding device has a retaining device. This means that the number of retaining devices preferably also corresponds to the number of holding devices.

The flowable sterilization medium is preferably H₂O₂. However, another substance such as peracetic acid can also be used, and it would also be possible to use sterilizing radiation for sterilizing said containers instead of a flowable, i.e., in particular liquid or gaseous, sterilization medium, for example UV radiation, laser radiation, or electron radiation. The applicant therefore reserves the right to claim protection for such configurations as well.

In a further advantageous embodiment, the apparatus comprises a treatment room within which the plastics material containers are or can be transported during their sterilization.

Preferably, the plastics material containers can be transported along a circular or circular segment-shaped transport path.

Preferably, the application device is also – advantageously completely – arranged within the treatment room. In a further advantageous embodiment, at least one, preferably at least two, further transport devices, in particular transport starwheels, are also arranged within the treatment room.

Preferably, the treatment room is configured as a cleanroom, within which sterile conditions can be maintained.

Particularly preferably, the holding devices, in particular the gripping devices (of the holding devices), each form transport pockets which are each suitable and intended for receiving a plastics material container or a (region of a) mouth portion of a plastics material container.

Preferably, the gripping devices are configured in such a way that they support the containers, in particular the plastics material preforms, in the longitudinal direction thereof. In particular, the containers, in particular the plastics material preforms, are supported by their support rings.

In a further preferred embodiment, the application device is arranged in such a way that it substantially and/or at least also allows the application of the flowable sterilization medium to the interior of the plastics material containers. Thus, it is possible for the sterilization medium to initially be applied to the interior of the plastics material containers, in particular plastics material preforms, and subsequently also to outer portions of the plastics material container, in particular plastics material preform, such as, in particular, a threaded and/or mouth region.

In particular, the application device is arranged above the plastics material containers and/or above the transport path of the plastics material containers.

Preferably, the application device has a plurality of application nozzles which are suitable and intended to apply the sterilization medium to the interior of the plastics material containers. Preferably, these application devices each have the same distance and/or the same pitch from one another as the holding devices.

In a preferred embodiment, the application device and/or these application nozzles is/are arranged stationary and the plastics material containers move relative to these application nozzles.

In a further preferred embodiment, the application devices and/or application nozzles move together with the plastics material containers. Preferably, the application nozzles are rigidly connected to the transport device, for example a transport starwheel, and move with each plastics material container, in particular each plastics material preform.

In a further preferred embodiment, the apparatus has a supply device which supplies the plastics material containers to the transport device. This supply device is particularly preferably a so-called pitch varying starwheel, which is suitable and intended to change the pitch between the plastics material containers transported thereby.

Particularly preferably, this supply device has a plurality of holding devices which are suitable and intended for holding the containers, in particular the plastics material preforms. Preferably, these holding devices each have holding elements that are suitable and intended to hold and/or fix the individual containers in a mouth region and in particular in a region above the support ring of the plastics material containers, in particular the plastics material preforms. This allows a simple transfer to the holding devices of the transport device.

It is therefore proposed that the plastics material containers, in particular the plastics material preforms, are introduced into the transport device or the individual holding devices and also removed therefrom, in particular by the supply device, which is in particular a pitch varying starwheel.

Particularly preferably, the apparatus also comprises a discharge device which is suitable and intended for discharging plastics material containers that are transported by the transport device and are also advantageously already sterilized from the transport device again.

This discharge device can preferably also be a pitch varying starwheel. However, it would also be conceivable that only one pitch varying starwheel is provided, which both supplies the plastics material containers to the transport device and discharges the plastics material containers from the transport device.

Preferably, disinfection takes place in the transport device, which is preferably configured as a pocket starwheel, in particular by gaseous H₂O₂.

The transport device, in particular the individual holding devices, particularly preferably grips each of the plastics material containers below their support ring.

Particularly preferably, the pitch between two successive holding devices, in particular between the recesses or pockets formed by the holding devices, is smaller than 2.5 times the support ring diameter of the plastics material preforms, preferably smaller than twice the support ring diameter, and preferably smaller than 1.5 times the support ring diameter.

In a preferred embodiment, the recess or receiving pocket has an opening, and in particular a semicircular opening. Particularly preferably, this opening is tangential, in particular over the center of the plastics material container and/or plastics material preform to be held, and in particular over the center of the portion of the plastics material container to be held.

Preferably, the plastics material preform or the plastics material container is secured against falling out by a sliding or movable element, in particular the aforementioned retaining device.

The present invention is further directed to a transport device for transporting containers, in particular plastics material containers, in particular plastics material preforms. The transport device has a transport carrier that is movable and in particular rotatable about a predefined axis of rotation, on which carrier a plurality of holding devices for holding the containers are arranged.

According to the invention, the holding devices each have (at least) one gripping device which is suitable and intended to contact the plastics material containers at least in a first portion of the plastics material containers.

Furthermore, the holding device and preferably the holding devices each has/have a retaining device which is suitable and intended to contact the plastics material containers in or on a second portion of the plastics material containers, wherein the gripping device is preferably fixed relative to the transport device (and/or the container to be transported) and the retaining device is movable relative to the transport device in order to thereby hold the containers on the first gripping device in order to thereby hold the containers on the gripping device, wherein the retaining device (64) has a retaining element that is movable between a retaining position and a release position, wherein, in the retaining position, the retaining element holds the container, in particular the mouth region of the container (10), on the gripping device and the retaining element, and, in the release position, the container can exit from a recess formed in the first gripping device, in particular without contacting the retaining element.

It is possible that a recess is formed by the gripping device and a further gripping device, or by the gripping device and the retaining device. The gripping device itself may also have a recess against which said portion of the plastics material container can rest.

It should be noted in this context that the gripping arm or gripping arms do not necessarily have to clampingly grip the containers. Therefore, the gripping arms can also be called holding arms. It is also conceivable that the containers, in particular the plastics material preforms, have (a small amount of) lateral play in the gripping devices.

The retaining device comprises a retaining element that can move between a retaining position and a release position, wherein, in the retaining position of the retaining element, the container, in particular a mouth region of the container, is held and/or retained between the gripping arms or holding arms, and, in the release position, the container, in particular the plastics material preform, can exit from the recess formed between the gripping arms, in particular without contacting the retaining element.

In particular, a retaining element that can move relative to the gripping arm(s) is proposed, which is adjustable and/or switchable between at least two positions. Preferably, the retaining element is pivotable with respect to a pivot axis which extends in the longitudinal direction or parallel to the longitudinal direction of the plastics material containers to be held.

In a further preferred embodiment, the retaining element is in the form of a gripping arm and/or the retaining element has a recess which can also be placed against the mouth region of the plastics material preform.

Preferably, the retaining element has a first portion and a second portion angled relative to the first portion. Preferably, the second portion is configured as a cantilever. In particular, at least in the retaining position, the cantilever or the second portion extends substantially in the same direction as the gripping arms.

Preferably, the first and second portions are at an angle to each other which is between 60° and 120°, preferably between 70° and 110°, particularly preferably between 80° and 100° and particularly preferably at 90°.

In a further position, the retaining element can be pivoted about a predefined pivot axis between the retaining position and the release position. Preferably, the retaining element is pivotable by an angle greater than 10°, preferably greater than 15°. Preferably, the retaining element can be pivoted by an angle that is smaller than 90°, preferably smaller than 80°, preferably smaller than 70°, preferably smaller than 60°, preferably smaller than 50°, and preferably smaller than 40°.

This pivot axis particularly preferably extends in a direction that is perpendicular (or parallel) to a longitudinal direction of a container that is or is to be held by the holding devices. Preferably, this pivot axis extends at an angle relative to a longitudinal direction of a container that is or is to be held by the holding device.

The pivot axis is particularly preferably located in a horizontal direction if the container is held in an upright or vertical orientation.

In a further preferred embodiment, when the container, in particular the plastics material container, is vertically oriented, the pivot axis runs above the gripping device or holding device, preferably above a plastics material container held by the gripping device.

However, it would also be possible for the retaining element to be movable in a predefined direction, in particular in the longitudinal direction of the plastics material containers, in order to move it from the retaining position to the release position (and vice versa).

In a further preferred embodiment, the transport device has at least one drive device for moving and in particular pivoting the retaining element(s).

Preferably, this drive device has a guide cam that is in particular stationary and relative to which guide rollers roll, wherein at least one guide roller is preferably assigned to each holding device and/or each retaining element. In this way, the guide cam drive can cause the retaining element to pivot and/or move between the retaining position and the release position.

However, other drive systems would also be conceivable, for example electric motor drives.

In a further preferred embodiment, the transport device has a switching means which is suitable for switching the retaining device and/or the retaining element between the retaining position and the release position. Particularly preferably, at least one magnetic fixing means is provided which is suitable and intended to fix the retaining device and/or the retaining element in one of the two positions (at least temporarily).

In other words, the transport device preferably has at least one magnetic fixing means which is suitable and intended to fix the retaining device and/or the retaining element in (at least) one of the two positions, in particular fixing it temporarily. Preferably, the gripping clamp is also switched between a closed state (in which a container is held) and an open state (in which no container is held) by movement of the retaining means.

In the prior art, spring means are usually used to hold the gripping clamps in an open or closed state. Within the scope of the invention, it is proposed to instead provide one or several magnetic means which hold the gripping clamp in at least one of the two states (or to hold the retaining device and/or the retaining element in one of the two positions).

Particularly preferably, the magnetic fixing means has at least one permanent magnet, wherein the at least one permanent magnet is preferably arranged on the retaining element or the retaining means and in particular on a pivotable retaining element and is preferably pivotable together with this retaining element.

Preferably, the switching means has at least one first magnetic element that is (mechanically) coupled to the holding device, wherein movement of this first magnetic element can cause the retaining element to switch between the first position and the second position, and the switching means preferably has at least one first magnetic actuating element that is arranged so as to be stationary with respect to the retaining element(s), preferably spatially stationary, past which the retaining element and/or the retaining device can be moved, wherein a magnetic force occurring as said retaining element and/or retaining device moves past said at least one first magnetic actuating element, in particular a non-contact magnetic force, and in particular magnetic repulsion (force) between the first magnetic element and the first magnetic actuating element causes the retaining element to switch between the first position and the second position, i.e., in particular between the retaining position and the release position.

In this embodiment, it is proposed that the switching is effected by a magnetic force and, in particular, a magnetic repulsion. In this way, it is possible for the element to be accordingly switched even at very high operating speeds. In addition, this type of drive is free from mechanical wear and tear, since the magnetic force is applied without contact. In addition, this type of switching can be performed more quietly.

Particularly preferably, at least one of the aforementioned states is an end state of a mechanical movement of the retaining device or the retaining element. In a further preferred embodiment, both positions are end positions of a mechanical movement of the retaining device or the retaining element. In this way, the magnetic repulsion forces the retaining device or the retaining element into a mechanical end state (in relation to a movement path of the retaining element).

These embodiments are particularly advantageous when the retaining device or retaining element is a gripping arm of a gripping clamp, as described in more detail below.

In a further preferred embodiment, the transport device has a further fixing means which is suitable and intended to fix the retaining device and/or the retaining element in the other of the two positions.

In a further advantageous embodiment, the switching means has a switching cam that is mechanically coupled to the retaining element or the retaining device and is preferably switchable by a switching means, in particular a stationary switching means. Preferably, this switching cam is rotatable or pivotable. This additional switching means can also be a magnetic element, and in particular a permanent magnet.

Furthermore, it is possible that an element of the switching cam engages in a recess which is formed in the retaining element. In this way, the retaining element is moved by the Rotation of the switching cam.

It would therefore be conceivable for the drives to be contactless, for example magnetically.

In addition, it would also be conceivable for control via an upstream and/or downstream (transport) starwheel. This starwheel could, for example, accordingly contact the retaining elements and thus control them.

In a further preferred embodiment, the holding device or each of the holding devices is/are arranged separately and/or in isolation on the transport carrier. In particular, the holding devices can be removed from the carrier independently of each other or arranged independently of each other on the transport carrier.

In a further preferred embodiment, the gripping arms are formed in one piece. In particular, the gripping devices are passive gripping devices, i.e., they can preferably not be operated themselves or remain permanently in the same gripping position. In this way, the apparatus can be configured more simply.

In a further advantageous embodiment, the gripping arm or each of the gripping arms has/have a curved portion and/or form a curved portion (referred to above as a recess) and in particular a portion that is curved in the shape of a circular segment. A region of the held container, in particular a region of the held plastics material preform, can preferably rest against this circularly curved portion.

In addition, the gripping arms preferably each have a straight portion that adjoins the curved portion. Particularly preferably, overall the gripping arms form a semicircular or almost semicircular portion.

Particularly preferably, the two gripping arms together form a U-shaped recess, wherein the recess is in particular semicircular on a side facing the axis of rotation of the transport carrier.

Preferably, the straight portions run parallel to each other.

The containers, in particular plastics material preforms, are particularly preferably insertable into the recess without resistance.

By providing the retaining element, it is possible to do without the guide arc that is usually present in sawtooth starwheels, for example. The applicant has found that, in particular when transporting plastics material preforms, the weight ratios or the center of gravity are such that the gripping device, in conjunction with the retaining element, is also sufficient to securely hold the plastics material preforms even at higher rotational speeds of the rotatable transport carrier.

Particularly preferably, the retaining element is configured in such a way that the cantilever also contacts (in particular with its underside) or almost contacts the containers, in particular the plastics material preforms. This also prevents the containers or plastics material preforms from tipping, for example due to the rotational force.

It is possible and preferred that the containers, in particular the plastics material preforms, are held with a slight amount of play by the interaction of the gripping devices and the retaining devices.

In a further advantageous embodiment, the retaining elements each have an opening which is positioned above the mouth of the plastics material containers or the plastics material preforms in the retaining position. This opening allows, for example, the plastics material preforms to be sterilized. Application nozzles can also be guided through these openings.

Thus, the recess or the, in particular each, receiving pocket of the individual holding devices preferably has a semicircular opening that is extended tangentially, in particular over the center of the plastics material preforms.

As mentioned, the containers, in particular the plastics material preforms, are secured against falling out by a holding element, referred to above as a retaining element. As mentioned above, this retaining element can be moved vertically or can preferably be pivoted in from above. As mentioned above, the pivot axis or axis of rotation of this retaining element lies above an upper edge of the containers, in particular the plastics material preforms.

As mentioned above, a sterilization medium can be supplied through a fixed nozzle, in particular from above.

The present invention further relates to a holding device for holding containers, in particular plastics material containers, in particular plastics material preforms, wherein the holding device has (at least) one gripping device which is suitable and intended to contact the plastics material containers at least in a first portion of the plastics material containers in order to thereby hold the containers on the first gripping device, and furthermore a retaining device is provided which is suitable and intended to contact the containers in a second portion of the containers, wherein the gripping device is fixed relative to the holding device and/or relative to the container to be held, and the retaining device is movable and in particular pivotable relative to the holding device and/or relative to the container to be held.

Furthermore, the retaining device has a retaining element that can move between a retaining position and a release position, wherein, in the retaining position, the retaining element holds the container, in particular the mouth region of the container, on the gripping device and preferably the retaining element, and, in the release position, the container can exit from a recess formed in the first gripping device, in particular without contacting the retaining element.

Preferably, the gripping device is fixed relative to the container and the retaining device is movable relative to the container (to be held).

In a further advantageous embodiment, the retaining device has a retaining element that can move between a retaining position and a release position, wherein, in the retaining position of the retaining element holds the container, in particular in the mouth region of the container, between the gripping device and a further gripping arm, and, in the release position, the container, in particular the plastics material preform, can exit from a recess formed between the gripping device and the further gripping arm, in particular without contacting the retaining element.

Preferably, the containers, in particular plastics material preforms, are arranged vertically, with the mouth of the containers, in particular plastics material preforms, arranged above a main body of the containers, in particular plastics material preforms.

Particularly preferably, the holding device has a fastening element which allows the holding device to be attached to a carrier and in particular a rotatable carrier. Preferably, this fastening element allows for manual and/or tool-free assembly and/or disassembly of the holding device on/from the transport carrier.

In a further preferred embodiment, the retaining element also forms a contact and/or cover portion which, in the position of the retaining element, either contacts the mouth edge of the container, in particular the plastics material preform, or is arranged directly adjacently thereto.

This contact or cover portion ensures that the plastics material preform held by the holding device cannot be pivoted, for example, under the influence of centrifugal force.

The retaining element, in its retaining position, is therefore particularly preferably also used as a hold-down element to hold down the container, in particular the plastics material preform. Preferably, the hold-down element also prevents the container, in particular the plastics material preform, from escaping the gripping device upward in its longitudinal direction, at least in the retaining position.

Particularly preferably, a distance between the cover portion and the mouth edge of the container is less than 1 cm, preferably less than 0.5 cm, preferably less than 0.4 cm, preferably less than 0.3 cm, preferably less than 0.2 cm, and particularly preferably less than 0.1 cm.

In addition, it would also be possible for the retaining element to hold the support ring from above in the retaining position, or to be arranged above the support ring, so that the plastics material preform thus cannot escape from the holding devices.

The retaining element could also be configured as a clamp or gripping arm. In this case, it could be ensured that substantially the entire mouth of the containers and the plastics material preforms remains accessible for sterilization.

In a further preferred embodiment, the holding device is suitable and intended for holding and/or transporting plastics material preforms, in particular in the manner described above.

The present invention is further directed to a method for treating and in particular sterilizing containers, in particular plastics material preforms. In this process, a transport device, which preferably has a transport carrier that can rotate about a predefined axis of rotation and on which a plurality of holding devices for holding the plastics material containers is or will be arranged, transports the containers along a predefined transport path.

Preferably, the holding devices each have a gripping device which contacts the plastics material containers at least in a first portion of the plastics material containers, and a retaining device which contacts the containers in a second portion of the containers, wherein the first portion is spaced apart from the second portion, and wherein the gripping device is fixed relative to the transport device and the retaining device is movable relative to the transport device, wherein the retaining device has a retaining element that can move between a retaining position and a release position, wherein, in the retaining position, the retaining element holds the container, in particular the mouth region of the container, on the gripping device, and, in the release position, the container can exit from a recess formed in the first gripping device, in particular without contacting the retaining element.

In a further preferred embodiment, the holding devices each have a first gripping device with two gripping arms (and/or a second gripping arm next to the first gripping device), wherein at least a first portion of the containers, in particular the plastics material containers, is received between the gripping device and the second gripping arm, and wherein the retaining device contacts each of the containers in a second portion of the containers, which is spaced apart from the first portion in a longitudinal direction of the containers, in order to thereby hold the containers between the gripping device and the gripping arm.

The retaining device has a retaining element that can move between a retaining position and a release position, wherein, in the retaining position of the retaining element holds the container, in particular the plastics material container, in particular the plastics material preform, in particular the mouth region of the plastics material preform, between the gripping arms (or between the gripping device and the gripping arm) and wherein in a release position, the container, in particular the plastics material container, in particular the plastics material preform, can exit from a recess formed between the gripping arms (or between the gripping device and the gripping arm), in particular without contacting the retaining element.

In particular, the retaining element holds the container, in particular the plastics material containers, on the radial outer circumference thereof. However, it would also be possible for it to be held via an upper edge or the mouth region of the container. However, it would also be possible for the retaining element to hold the container at the same height as the gripping device. In this case, the retaining element preferably also acts as a second gripping arm that interacts with the gripping device.

In a further advantageous method, a flowable sterilization medium and/or stabilizing radiation is/are applied to the containers, in particular the plastics material preforms, during their transport. The interior of the plastics material preforms is particularly preferably also sterilized.

The present invention in particular ensures simpler and more robust handling, in particular for plastics material preforms in a treatment module.

### BRIEF DESCRIPTION OF THE DRAWINGS

Further advantages and embodiments emerge from the accompanying drawings, in which:

FIG. 1 shows a rough schematic representation of an apparatus in particular for sterilizing plastics material preforms;

FIG. 2 shows a detailed view of the apparatus according to the invention; and

FIG. 3 shows a top view of an apparatus or a portion in a further embodiment according to the invention.

### DETAILED DESCRIPTION OF THE INVENTION

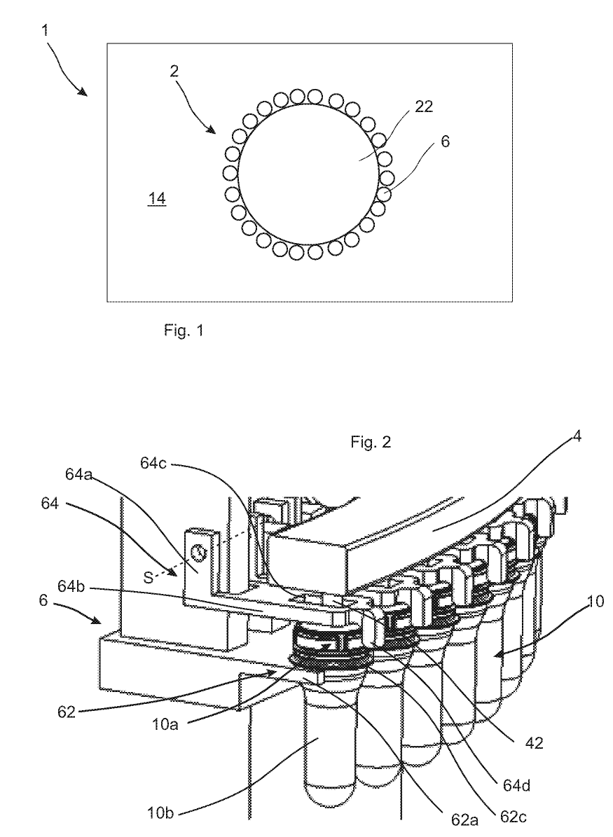

FIG. 1 shows a rough schematic representation of an apparatus 1 for sterilizing plastics material preforms in a first embodiment. A transport device 2 is provided within a treatment room 14 for transporting the plastics material preforms (not shown). This transport device 2 has a rotatable transport carrier 22 on which a plurality of holding devices 6 of the type described above are arranged, preferably at equal distances.

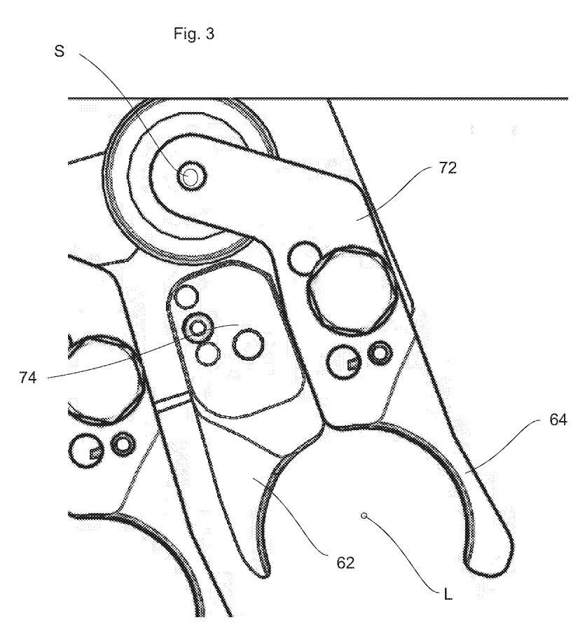

FIG. 2 shows a partial view of the transport device 2 in a first embodiment. It can be seen that a plurality of plastics material preforms 10 can be held here, each plastics material preform 10 having a mouth portion 10b and a main body 10b.

Reference sign 6 designates a holding device, which serves here to hold a single plastics material preform 10. This holding device 6 has a gripping device 62, which here has a gripping arm 62a and a second gripping arm (not shown). In a further embodiment as described above, the gripping device is a gripping arm which is fixed (in particular with respect to the plastics material preforms 10). A plastics material preform can be held or supported by this gripping device below its support ring 10c.

Reference sign 64 designates a retaining element which has a first portion 64a and a second portion or cantilever 64b. In a further embodiment illustrated below, the retaining element is configured as a gripping arm.

The retaining element 64 is pivotable with respect to a pivot axis S, namely from a retaining position shown in FIG. 2 two to a release position. Reference sign 64d designates a projection of the retaining apparatus or a retaining projection which is suitable for retaining plastics material preforms 10 so that they cannot leave the gripping device.

Reference sign 64c indicates an opening which is arranged in the cantilever 64b. For example, an application nozzle 42 of an application device, designated as a whole by reference sign 4, can be guided through this opening.

FIG. 3 shows a further embodiment of a holding device. A gripping device 62 is provided, which is formed as a gripping arm in this case. Reference sign 64 designates the retaining device which is pivotable with respect to a pivot axis S. In this case, this pivot axis S extends perpendicularly to the plane of the drawing and also parallel to the longitudinal direction of the plastics material preforms or plastics material containers (not shown). Preferably the pivot axis is at least 2 cm, preferably at least 3 cm, preferably at least 4 cm and particularly preferably at least 5 cm away from the (geometric) longitudinal axis of the plastics material preforms. Preferably, the pivot axis is at most 30 cm, preferably at most 25 cm, preferably at most 20 cm and particularly preferably at most 15 cm away from the (geometric) longitudinal axis of the plastics material preforms.

It can be seen that the gripping device 62 and the retaining device or retaining element collectively surround the plastics material container (not shown) at a circumferential angle greater than 180°, preferably greater than 190°, preferably greater than 200° and particularly preferably greater than 210°. Preferably, this angle is smaller than 320°, preferably smaller than 310°, preferably smaller than 300°, preferably smaller than 290°, preferably smaller than 280°, preferably smaller than 270° and particularly preferably smaller than 260°.

The position of the longitudinal axis L of the plastics material containers (not shown) is designated by the letter L. It can also be seen, as explained above, that the circumferential angle at which the retaining element surrounds the plastics material container is larger than the circumferential angle at which the gripping device surrounds the plastics material container.

Reference sign 72 designates a cantilever on which the retaining element 64 is arranged. Reference sign 74 designates a fastening element with which the gripping device 62 is stationary, but preferably adjustably, fastened to a carrier. This adjustability preferably makes it possible to adapt to different geometries of plastics material preforms or plastics material containers in general.

It is pointed out that all features which have been described with reference to the method are also correspondingly disclosed for the apparatus, which means in particular that the corresponding apparatus comprises devices which are suitable and intended for carrying out the particular methods. Furthermore, features which were described with reference to the apparatus are also correspondingly applicable to the method or methods. This means that the methods are carried out using the corresponding apparatus features.

It should also be noted that all the features described with reference to the apparatus for treating plastics material containers also accordingly to the transport apparatus and the holding device. The same also applies to features of the transport device and the holding device, which are accordingly also disclosed for the apparatus for treating plastics material containers.

The applicant reserves the right to claim all features disclosed in the application documents as essential to the invention, provided that they are novel over the prior art individually or in combination. It is further pointed out that features which can be advantageous in themselves are also described in the individual figures. A person skilled in the art will immediately recognize that a particular feature described in a figure can be advantageous even without the incorporation of further features from this figure. Furthermore, a person skilled in the art will recognize that advantages can also result from a combination of several features shown in individual or in different figures.

1. An apparatus for treating containers, comprising a transport device for transporting the plastics material containers along a predefined transport path and comprising at least one application device suitable and intended for applying a flowable sterilization medium and/or a coating and/or electromagnetic radiation to the plastics material containers transported by the transport device, wherein the transport device has a movable transport carrier on which a plurality of holding devices for holding the plastics material containers are arranged,
wherein
the holding device comprises a gripping device which is configured for contacting the plastics material containers at least in a first portion of the plastics material containers, and a retaining device which is configured for contacting the plastics material containers in a second portion of the plastics material containers, wherein the first portion is spaced apart from the second portion, wherein the gripping device is fixed relative to the transport device and the retaining device is movable relative to the transport device.

2. The apparatus according to claim 1,
wherein
the apparatus has a treatment room within which the containers are transported during their sterilization.

3. The apparatus according to claim 1,
characterized in that
the application device is arranged in such a way that it substantially enables the application of the flowable sterilization medium to the interior of the containers.

4. A transport device for transporting containers, wherein the transport device has a transport carrier that is configured to move about a predefined axis of rotation and on which a plurality of holding devices for holding the containers are arranged,
wherein
the holding devices comprise a gripping device which is configured for contacting the plastics material containers at least in a first portion of the plastics material containers, and a retaining device which is configured for contacting the containers in a second portion of the containers, wherein the first portion is spaced apart from the second portion, wherein the gripping device is fixed relative to the transport device and/or to the container to be transported, and the retaining device is movable relative to the transport device and/or to the container to be transported in order to thereby hold the containers on the gripping device, wherein the retaining device comprises a retaining element which is configured to move between a retaining position and a release position, wherein, in the retaining position, the retaining element holds the container on the gripping device, and, in the release position, the container can exit from a recess formed in the gripping device together with the retaining element.

5. The transport device according to claim 4,
wherein
the retaining element can be pivoted about a predefined pivot axis between the retaining position and the release position.

6. The transport device according to claim 4,
wherein
the transport device has at least one drive device for moving the retaining elements.

7. The transport device according to claim 4,
wherein
the transport device has a switching device which is configured for switching the retaining device or retaining element between the retaining position and the release position.

8. The transport device according to claim 7,
Wherein the retaining element comprises at least one magnet.

9. The transport device according to claim 8, wherein the at least one magnet comprises a permanent magnet.

10. The transport device according to claim 4,
wherein
the holding devices are arranged separately on the carrier.

11. The transport device according to claim 4,
wherein
the gripping device surrounds the plastics material container to be held and/or the first portion to be held at a first predefined circumferential angle, and the retaining device surrounds the plastics material container to be held at a second predefined circumferential angle.

12. A holding device for holding containers, wherein the holding device comprises a gripping device which is configured for contacting the plastics material containers at least in a first portion of the plastics material containers, and a retaining device which is configured for contacting the containers in a second portion of the containers in order to thereby hold the containers on the gripping device, wherein the first portion is spaced apart from the second portion, wherein the retaining device comprises a retaining element that is configured to move between a retaining position and a release position, wherein, in the retaining position, the retaining element is configured to hold the container on the gripping device and the retaining element, and, in the release position, permit the container to exit from a recess formed in the gripping device.

13. The holding device according to claim 12,
wherein
the retaining element has an element which is in contact with the container when in the retaining position.

14. The holding device according to claim 12,
wherein
the first portion and the second portion are spaced apart from one another in the longitudinal direction of the containers and/or in the circumferential direction of the containers.

15. A method for treating containers, wherein a transport device comprising a transport carrier that is configured to rotate about a predefined axis of rotation and on which a plurality of holding devices for holding the containers are arranged transports the containers along a predefined transport path,
wherein the holding devices each have a gripping device which contacts the plastics material containers at least in a first portion of the plastics material containers and a retaining device which contacts containers in a second portion of the containers, wherein the first portion is spaced apart from the second portion and wherein the gripping device is fixed relative to the transport device and/or to the container and the retaining device is movable relative to the transport device and/or to the container, wherein the retaining device has a retaining element that moves between a retaining position and a release position, wherein, in the retaining position, the retaining element holds the container on the gripping device, and, in the release position, the container can exit from a recess formed in the gripping device.

16. The method according to claim 15,
wherein
a flowable sterilization medium or sterilizing radiation is applied to the plastics material preforms during their transport.
